(19) United States
(12) Patent Application Publication
(10) Pub. No.: US 2023/0078479 A1
(43) Pub. Date: Mar. 16, 2023

(54) REAL-TIME MONITORING DEVICE FOR HUMAN BODY

(71) Applicant: SiriuXense Co., Ltd., Hsinchu City (TW)

(72) Inventors: Fu-Ji Tsai, New Taipei City (TW); Ji-De Huang, Hsinchu City (TW); Ju-Yu Hung, Taichung City (TW); Chen-I Kuo, Taipei City (TW); Chih-Chien Lu, Taoyuan City (TW)

(73) Assignee: SiriuXense Co., Ltd., Hsinchu City (TW)

(21) Appl. No.: 17/892,121

(22) Filed: Aug. 22, 2022

Related U.S. Application Data
(60) Provisional application No. 63/243,108, filed on Sep. 11, 2021.

(51) Int. Cl. A61B 5/0205; A61B 5/00

(52) U.S. Cl. CPC A61B 5/02055; A61B 5/002; A61B 5/746; A61B 5/6838; A61B 2503/04; A61B 2562/0271; A61B 2562/0204

(57) ABSTRACT

A real-time monitoring device for human body is disclosed. The real-time monitoring device includes a sensor module and a processor module, wherein the sensor module is adopted for contacting a human body like a baby's, so as to conduct a sensing work. The processor module is coupled to the sensor module for receiving a body temperature sensing signal, a first sound signal and a body activity sensing signal, and is configured for generating a second sound signal by collecting a sound emitted from the body. According to the present invention, the processor module is configured for determining whether the baby has a physical condition after applying processing and analyzing the body temperature sensing signal, the first sound signal, the second sound signal, and the body activity sensing signal. Moreover, the processor is also configured for to estimating physiological parameters of the baby.

### CROSS-REFERENCE TO RELATED APPLICATIONS

This application claims benefits of U.S. Provisional Patent Application Ser. No. 63/243,108 for “Real-time monitoring system for babies”, filed Sep. 11, 2021. The contents of which are hereby incorporated by reference in its entirety for all purposes.

### BACKGROUND OF THE INVENTION

### 1. Field of the Invention

The present invention relates to the technology field of an electronic device configured for monitoring physical conditions and/or physiological parameters from a human body, and more particularly to a real-time monitoring device for human body.

### 2. Description of the Prior Art

There have been a variety of baby monitoring systems proposed. For example, U.S. Pat. No. 9,402,596B1 discloses a bowel sound analysis system, U.S. Pat. No. 8,094,013B1 discloses a baby monitoring system, U.S. Pat. No. 8,461,996B2 discloses an infant monitor, and U.S. patent publication No. 2005/0195085A1 discloses a wireless monitoring system of diaper wetness, motion, temperature and sound.

According to the disclosures of U.S. Pat. No. 9,402,596B1, the bowel sound analysis system is configured for merely determining a health condition of the intestinal tract of a baby by collecting and analyzing intestinal motility signals, and fails to simultaneously measure physiological parameters (e.g., heart rate and respiratory rate) and/or determine physical conditions of the baby. On the other hand, according to the disclosures of U.S. Pat. No. 8,094,013B1, the baby monitoring system is configured for measuring breath rate and determining body orientation of a child, and is not allowed for being used to monitor at least one body activity like excretion. Furthermore, according to the disclosures of U.S. Pat. No. 8,461,996B1, the infant monitor is configured for measuring movements of an infant's body, so as to monitor breathing, heartbeat, body temperature, and the like. However, the infant monitor still fails to monitor or determine at least one body activity (e.g., excretion) of the infant.

According to above descriptions, it is understood that there are still rooms for improvement in the conventional baby monitoring system. In view of this fact, inventors of the present application have made great efforts to make inventive research and eventually provided a real-time monitoring device for human body (infant's body).

### SUMMARY OF THE INVENTION

The primary objective of the present invention is to disclose a real-time monitoring device for simultaneously measuring physiological parameters (e.g., heart rate and respiratory rate) and determining physical conditions of a human body like baby's. The real-time monitoring device comprises a sensor module and a processor module, wherein the sensor module is adopted for contacting a body of a baby, so as to measure a body temperature from the body, collect a sound emitted from the body, and monitor a movement and/or a vibration of the body, thereby generating a body temperature sensing signal, a first sound signal and a body activity sensing signal. On the other hand, the processor module is coupled to the sensor module, and is configured for generating a second sound signal after collecting the sound emitted from the body and an ambient sound, and then determining whether the baby has a physical condition after applying a signal analyzing process to the body temperature sensing signal, the first sound signal, the second sound signal, and the body activity sensing signal. The physical condition includes: excretion, abnormal heart rate (HR), abnormal respiration rate (RR), emission of abnormal bowel sounds, airway obstruction, going into a deep sleep, going into a light sleep, and going into a paradoxical sleep. Moreover, after processing and analyzing the first sound signal, the second sound signal and the body activity sensing signal, the processor module also estimates physiological parameters of the baby, including heart rate and respiration rate.

For achieving the primary objective mentioned above, the present invention provides an embodiment of the real-time monitoring device for human body, comprising:

a sensor module, comprising a first body and a first circuit assembly disposed in the first body, wherein the first circuit assembly comprises a first microphone, a temperature sensor and an inertial sensor; and

a processor module, comprising a second body and a second circuit assembly disposed in the second body, wherein the second circuit assembly comprises a second microphone, a microprocessor, a memory, and a wireless transmission interface;

wherein the first body is allowed to be contacted a human body by a body contacting surface thereof, and the memory storing an application program including instructions, such that in case the application program is executed, the microprocessor being configured for:
controlling the temperature sensor to measure a body temperature from the human body, thereby generating a body temperature sensing signal;
controlling the first microphone to collect a sound emitted from the human body, thereby generating a first sound signal;
controlling the inertial sensor to monitor a movement and/or a vibration of the human body, thereby generating a body activity sensing signal;
controlling the second microphone to collect said sound emitted from the human body and an ambient sound, thereby generating a second sound signal;
judging whether the human body has at least one physical condition by comparing the first sound signal with the second sound signal; and
analyzing the body temperature sensing signal, the first sound signal, the second sound signal, and the body activity sensing signal, so as to determine said physical condition includes at least one selected from a group consisting of excretion, abnormal heart rate (HR), abnormal respiration rate (RR), emission of abnormal bowel sounds, airway obstruction, going into a deep sleep, going into a light sleep, and going into a paradoxical sleep.

In one embodiment, the application program consists of a plurality of subprograms, and the plurality of subprograms comprising:

a first subprogram, being compiled to be integrated in the application program by one type of programming language, and including instructions for configuring the microprocessor to control the temperature sensor to measure the body temperature from the human body;

a second subprogram, being compiled to be integrated in the application program by one type of programming language, and including instructions for configuring the microprocessor to control the first microphone and the second microphone to collect the sound emitted from the human body;

a third subprogram, being compiled to be integrated in the application program by one type of programming language, and including instructions for configuring the microprocessor to control the inertial sensor to monitor the movement and/or the vibration of the human body;

a fourth subprogram, being compiled to be integrated in the application program by one type of programming language, and including instructions for configuring the microprocessor to process the body temperature sensing signal, the first sound signal, the second sound signal, and/or the body activity sensing signal;

a fifth subprogram, being compiled to be integrated in the application program by one type of programming language, and including instructions for configuring the microprocessor to apply a signal synchronizing process to the body temperature sensing signal, the first sound signal, the second sound signal, and the body activity sensing signal according to four timestamps that are respectively contained in the body temperature sensing signal, the first sound signal, the second sound signal, and the body activity sensing signal; and

a sixth, being compiled to be integrated in the application program by one type of programming language, and including instructions for configuring the microprocessor to judge whether the human body has at least one physical condition and then determine said physical condition.

In one embodiment, the plurality of subprograms further comprises:

a seventh subprogram, being compiled to be integrated in the application program by one type of programming language, and including instructions for configuring the microprocessor to calculate an estimated body temperature according to the body temperature sensing signal, and to estimate at least one physiological parameter of the human body by processing the first sound signal, the second sound signal and the body activity sensing signal; wherein the physiological parameter is selected from a group consisting of heart rate (HR) and respiration rate (RR).

In one embodiment, the plurality of subprograms further comprises:

an eighth subprogram, being compiled to be integrated in the application program by one type of programming language, and including instructions for configuring the microprocessor to judge whether there is a well contact between the first body and the human body by analyzing the body temperature sensing signal, the body activity sensing signal, a first frequency band and a second frequency band of the first sound signal.

In one embodiment, the plurality of subprograms further comprises:

a ninth subprogram, being compiled to be integrated in the application program by one type of programming language, and including instructions for configuring the microprocessor to generate a warning signal in case of there is existing said physical condition and/or at least one said physiological parameter exceeding a normal range, and then to transmit the warning signal to an electronic device through the wireless transmission interface.

In one embodiment, the electronic device is selected from a group consisting of signal transceiver device, tablet computer, cloud server, laptop computer, desktop computer, all-in-one computer, smart phone, smart watch, and smart glasses.

In one embodiment, the memory is selected from a group consisting of embedded flash (eFlash) memory, flash memory chip, hard drive (HD), solid state drive (SSD), and USB flash drive.

In one embodiment, the microprocessor is provided with an analog-to-digital (A/D) convertor therein, and the A/D convertor directly digitizes the first sound signal, digitizes the second sound signal using a first sampling rate, and digitizes the body activity sensing signal using a second sampling rate.

In one embodiment, the first sampling rate is not greater than 4 KHz, and the second sampling rate is not greater than 120 Hz.

In one embodiment, the first body has a first accommodation space for receiving the first circuit assembly therein, and a first cover is connected to a first opening of the first accommodation space so as to shield the first circuit assembly.

In one embodiment, an aperture is formed on a bottom of the first accommodation space, such that the first microphone is exposed out of the first body via the aperture.

In one embodiment, a circular recess is formed on the body contacting surface of the first body, and the circular recess has a depth and a diameter in a range between 4.5 mm and 20 mm, such that a ratio of the diameter to the depth is not greater than 6.

In one embodiment, a minimum value of the depth is 1.5 mm.

In one embodiment, the second body has a second accommodation space for receiving the second circuit assembly therein, and a second cover is connected to a second opening of the second accommodation space so as to shield the second circuit assembly.

In one embodiment, a body connecting member is connected between the first body and the second body, and the body connecting member is provided with an electrical connecting component therein, such that the first circuit assembly is coupled to the second circuit assembly through the electrical connecting component.

In one embodiment, the real-time monitoring device according to the present invention further comprises:

an article supporting unit, being disposed in the second accommodation space, and consisting of a platform and a plurality of supporting rods; wherein the platform is faced to a bottom of the second accommodation space, and the second circuit assembly being positioned in a space formed by the plurality of supporting rods and a bottom surface of the platform.

In one embodiment, the processor module further comprises:

a wireless charging module, being disposed on a top surface of the platform, and being coupled to the second circuit assembly; and

a battery, being coupled to the second circuit assembly.

In one practicable embodiment, the second body, the body connecting member and the first body are allowed to be fixed on a mounting kit, such that after disposing the mounting kit on an article that is worn on the human body, the first body being set to contact the human body by the body contacting surface thereof. Moreover, in case of the first body being set to contact the human body, a device fixing member is allowed to be used in further fixing the second body on the article.

In another one practicable embodiment, the second body and the first body are allowed to be connected with a device fixing member, such that the second body and the first body are allowed to be attached onto the human body through the device fixing member, thereby making the first body 11 contact the human body by the body contacting surface thereof.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1A shows a first stereo diagram of a real-time monitoring device for human body according to the present invention;

FIG. 1B shows a second stereo diagram of the real-time monitoring device;

FIG. 2 shows a diagram for describing an application of the real-time monitoring device;

FIG. 3A shows a third stereo diagram of the real-time monitoring device;

FIG. 3B shows a fourth stereo diagram of the real-time monitoring device;

FIG. 3C shows a fifth diagram of the real-time monitoring device;

FIG. 3D shows a sixth stereo diagram of the real-time monitoring device;

FIG. 4A shows a seventh stereo diagram of the real-time monitoring device;

FIG. 4B shows an eighth stereo diagram of the real-time monitoring device;

FIG. 5A shows a first exploded diagram of the real-time monitoring device;

FIG. 5B shows a second exploded diagram of the real-time monitoring device;

FIG. 6 shows a block diagram of a first microphone, a temperature sensor, an inertial sensor, a second microphone, a microprocessor, a memory, and a wireless transmission interface;

FIG. 7 shows a measured data graph of the body activity sensing signal, the body temperature sensing signal and the first sound signal;

FIG. 8 shows a FFT spectrogram of the first sound signal containing airway obstruction feature;

FIG. 9 shows a measured data graph of the first sound signal, the FFT spectrogram of the first sound signal, the second sound signal, the FFT spectrogram of the second sound signal, and body activity sensing signal;

FIG. 10 shows a measured data graph of the first sound signal, the FFT spectrogram of the first sound signal, the second sound signal, the FFT spectrogram of the second sound signal, and body activity sensing signal; and

FIG. 11 shows a measured data graph of hearth rate signal and respiration rate signal.

### DETAILED DESCRIPTION OF THE PREFERRED EMBODIMENTS

To more clearly describe a real-time monitoring device for human body according to the present invention, embodiments of the present invention will be described in detail with reference to the attached drawings hereinafter.

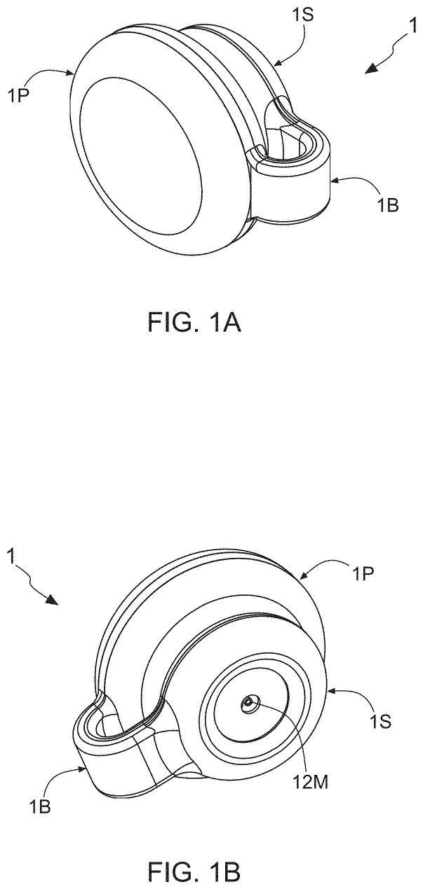

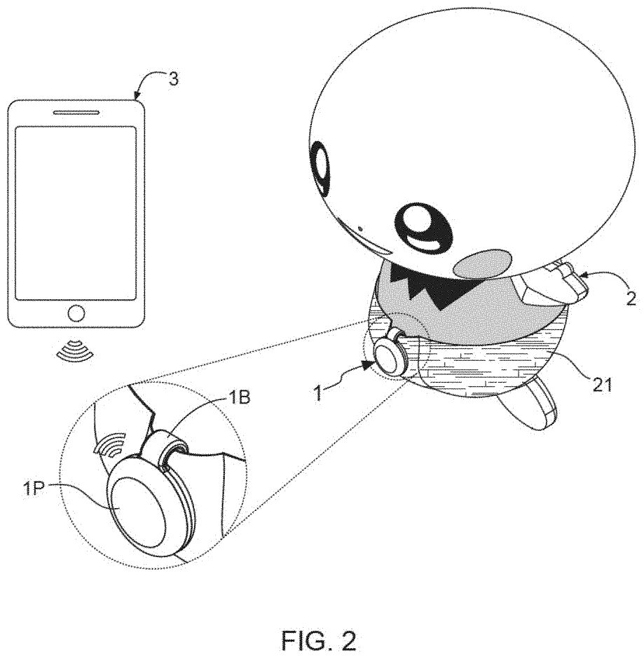

With reference to FIG. 1A and FIG. 1B, there are provided a first stereo diagram and a second stereo diagram of a real-time monitoring device for human body according to the present invention. The real-time monitoring device 1 is particularly designed for simultaneously measuring physiological parameters (e.g., heart rate and respiratory rate) and determining physical conditions of a human body like baby's. As FIG. 1A and FIG. 1B show, the real-time monitoring device 1 comprises a sensor module 1S, a processor module 1P and a body connecting member 1B connected between the sensor module 1S and the processor module 1P. In addition, FIG. 2 shows a diagram for describing an application of the real-time monitoring device. According to FIG. 1A, FIG. 1B and FIG. 2, it is understood that, the sensor module 1S, the processor module 1P and the body connecting member 1B are allowed to be fixed on a mounting kit 1K, such that the body connecting member 1B is bent to have a first curvature.

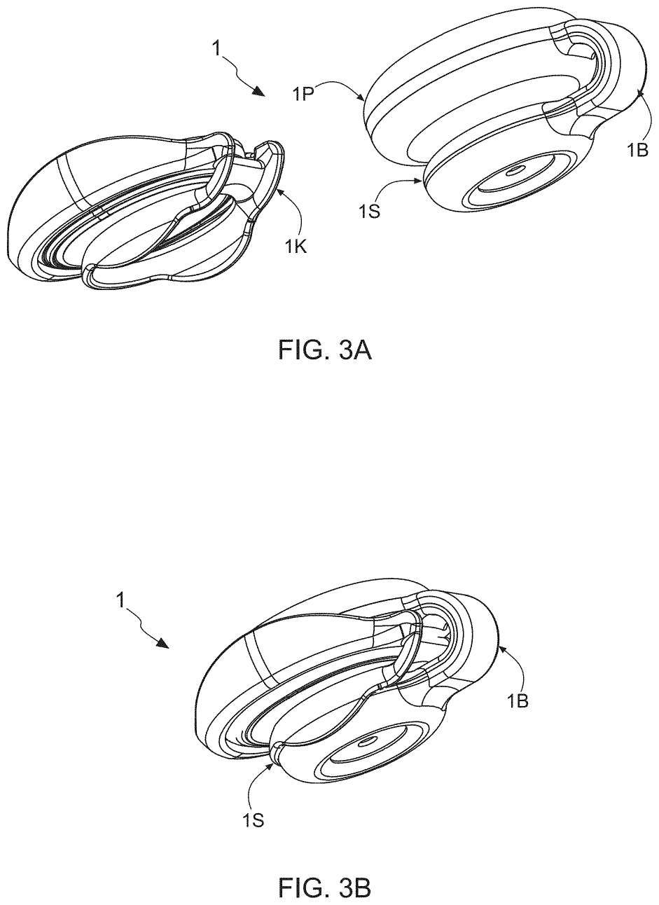

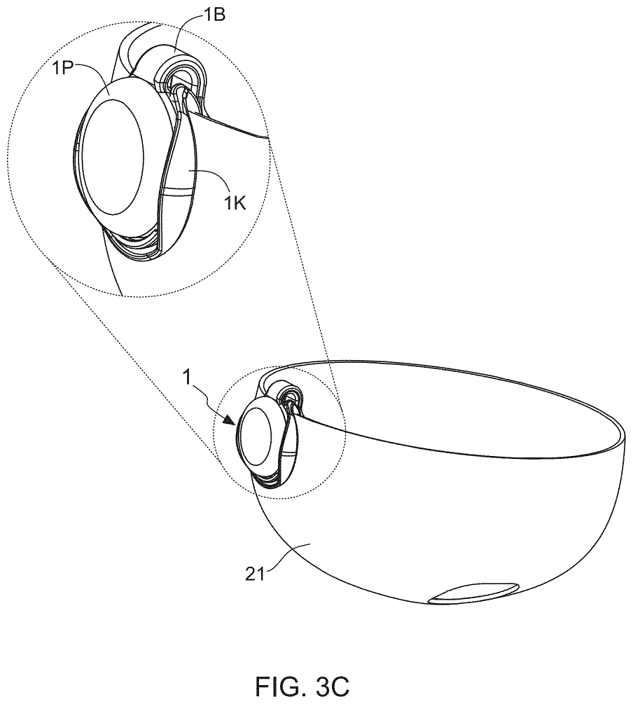

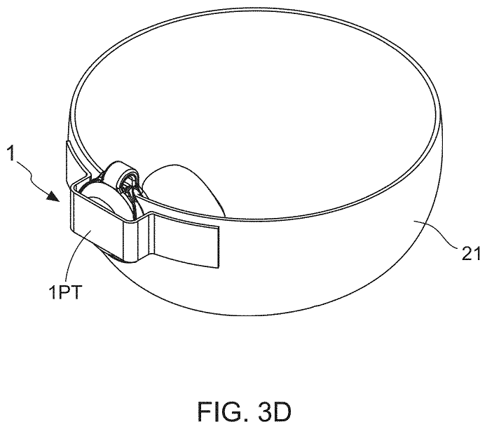

On the other hand, FIG. 3A, FIG. 3B and FIG. 3C show a third stereo diagram, a fourth stereo diagram and a fifth stereo diagram of the real-time monitoring device, respectively. When using this real-time monitoring device 1, the second body 13, the body connecting member 1B and the first body 11 are allowed to be fixed on the mounting kit 1K, such that it is able to next dispose the mounting kit 1K on an article that is worn on the human body, e.g., a diaper 21 worn on a baby 2. In such case, the real-time monitoring device 1 is hung on the top opening edge of the diaper 21 via the mounting kit 1K, such that the sensor module 1S is set to contact the human body (e.g., a body 2 of a baby) by a body contacting surface thereof. Furthermore, FIG. 3D illustrates a sixth stereo diagram of the real-time monitoring device. As FIG. 3D shows, in case of the sensor module 1S being set to contact the body 2, a first device fixing member 1PT is allowed to be used in further fixing the processor module 1P on an article of the body 2 (i.e., diaper 21).

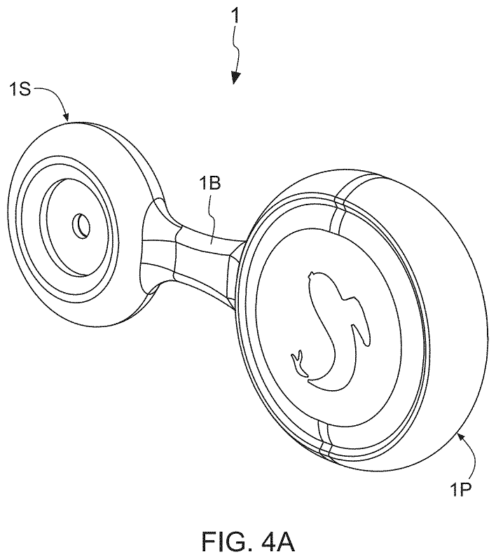

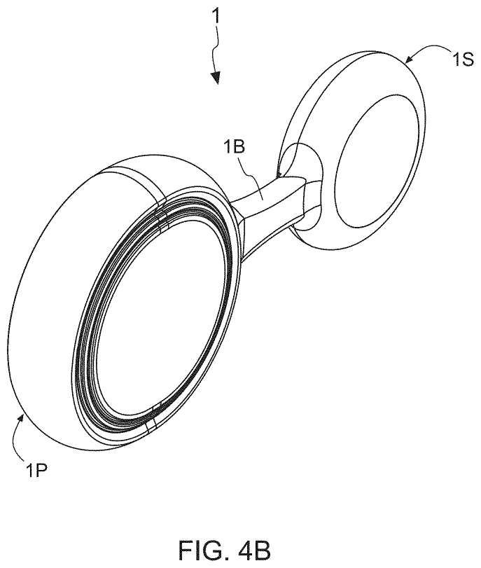

FIG. 4A and FIG. 4B illustrate a seventh stereo diagram and an eighth stereo diagram of the real-time monitoring device, respectively. In another practicable application, as FIG. 4A and FIG. 4B show, the real-time monitoring device 1 is allowed to be spread out, so as to make the body connecting member 1B has a second curvature smaller than the foregoing first curvature. In such case, the real-time monitoring device 1 is allowed to be connected with a second device fixing member (not shown), and then be attached onto the body 2 through the second device fixing member, thereby making the sensor module 1S contact the body 2 by the body contacting surface thereof.

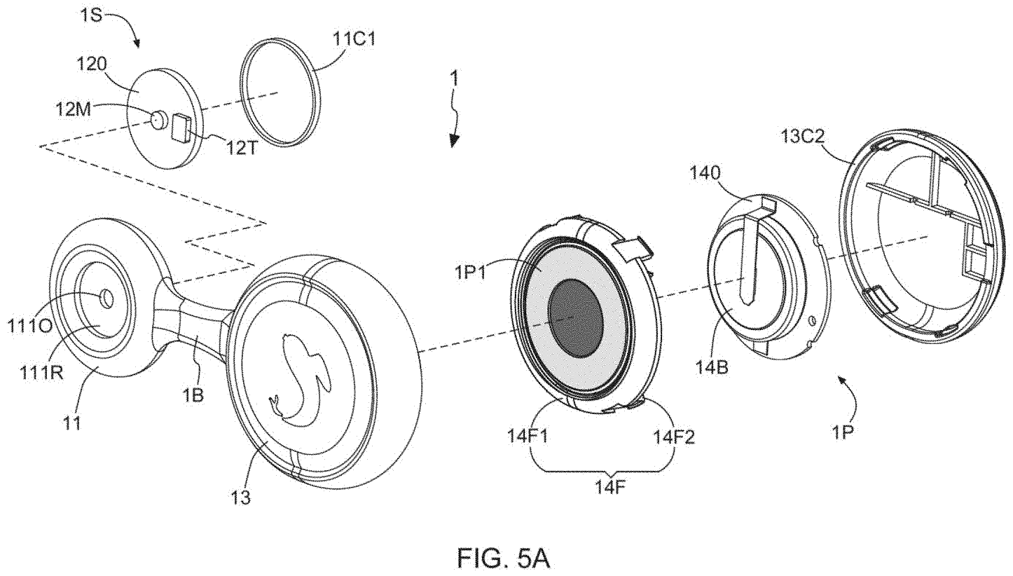

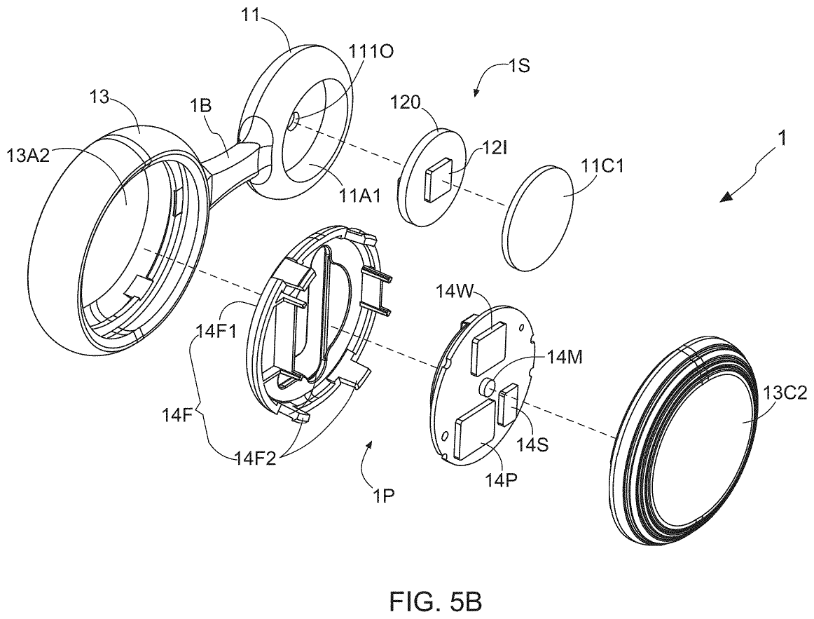

FIG. 5A and FIG. 5B illustrate a first exploded diagram and a second exploded diagram of the real-time monitoring device, respectively. As FIG. 5A and FIG. 5B show, the sensor module 1S comprises a first body 11 and a first circuit assembly disposed in the first body 11, of which the first circuit assembly comprises a first circuit board 120 and a first microphone 12M, a temperature sensor 12T and an inertial sensor 12I dispose on the first circuit board 120. On the other hand, the processor module 1P comprises a second body 13 and a second circuit assembly disposed in the second body 13, of which the second circuit assembly comprises a second circuit board 140 and a second microphone 14M, a microprocessor 14P, a memory 14S, and a wireless transmission interface 14W disposed on the second circuit board 140.

As described in more detail below, the first body 11 has a first accommodation space 11A1 for receiving the first circuit assembly therein, and a first cover 11C1 is connected to a first opening of the first accommodation space 11A1 so as to shield the first circuit assembly. Moreover, an aperture 111O is formed on a bottom of the first accommodation space 11A1, such that the first microphone 12M is exposed out of the first body 11 via the aperture 111O. On the other hand, the second body 13 has a second accommodation space 13A2 for receiving the second circuit assembly therein, and a second cover 13C2 is connected to a second opening of the second accommodation space 13A2 so as to shield the second circuit assembly. Particularly, the body connecting member 1B is connected between the first body 11 and the second body 13, and the body connecting member 1B is provided with an electrical connecting component therein, such that the first circuit assembly is coupled to the second circuit assembly through the electrical connecting component.

According to the present invention, a circular recess 111R is formed on the body contacting surface of the first body 11, and the circular recess 111R has a depth and a diameter in a range between 4.5 mm and 20 mm, such that a ratio of the diameter to the depth being not greater than 6. By such design, after the first body 11 is set to contact the human body (e.g., the baby's belly) by a body contacting surface thereof, the circular recess 111R helps the body contacting surface to well contact the skin of the baby's belly with high air tightness, thereby making an acoustic coupling path be formed between the first body 11 and a sound source portion of the baby (e.g., peritoneal cavity). It is worth further explaining that the human body is a low frequency resonator. Therefore, in case of there being a sound emitted by heart, lungs, respiratory tract, intestines, and/or excretion (i.e., the sound source portion), magnitude of the low frequency band of the sound would be amplified by the low frequency resonator, wherein said low frequency band includes sound signal falls below 25 Hz. Moreover, because there is an acoustic coupling path formed between the first body 11 and the sound source portion of the human body, the sound emitted by the human body is directly corrected by the first microphone 12M through the acoustic coupling path.

In a specific embodiment, the depth can be designed to have a minimum value of 1.5 mm. On the other hand, in case of the first body 11 being set to contact the human body by the body contacting surface thereof, the circular recess 111R is also allowed to prevent the aperture 111O (i.e., sound collecting hole for the first microphone 12M) from being plugged by the baby's belly. As described in more detail below, an article supporting unit 14F is disposed in the second accommodation space 13A2. As FIG. 5A and FIG. 5B show, the article supporting unit 14F consists of a platform 14F1 and a plurality of supporting rods 14F2, of which the platform 14F1 is faced to a bottom of the second accommodation space 13A2, and the second circuit assembly is positioned in a space formed by the plurality of supporting rods 14F2 and a bottom surface of the platform 14F1. Moreover, in the second body 13, there is a wireless charging module 1P1 disposed on a top surface of the platform 14F1 so as to be coupled to the second circuit assembly, and a battery 14B is coupled to the second circuit assembly. By such arrangements, it is allowed to put the real-time monitoring device 1 on a particularly-designed signal transceiver device, so as to make the second body 13 contact the signal transceiver device by a device contacting surface thereof. In such case, the signal transceiver device transmits electricity energy to the wireless charging module 1P1 through electromagnetic induction, such that the battery 14B is charged by the electricity energy.

As FIG. 1A, FIG. 1B, FIG. 2, FIG. 5A, and FIG. 5B show, the sensor module 1S is configured for measure a body temperature from the body 2, collect a sound emitted from the body 2, and monitor a movement and/or a vibration of the body 2, thereby generating a body temperature sensing signal, a first sound signal and a body activity sensing signal. On the other hand, the processor module 1P is coupled to the sensor module 1S, and is configured for generating a second sound signal after collecting the sound emitted from the body 2 and an ambient sound. As a result, after applying a signal analyzing process to the body temperature sensing signal, the first sound signal, the second sound signal, and the body activity sensing signal, the processor module 1P determines whether the body 2 has a physical condition or not. The physical condition includes: excretion, abnormal heart rate (HR), abnormal respiration rate (RR), emission of abnormal bowel sounds, airway obstruction, going into a deep sleep, going into a light sleep, and going into a paradoxical sleep. Moreover, after processing and analyzing the first sound signal, the second sound signal and the body activity sensing signal, the processor module 1P also estimates physiological parameters of the baby, including heart rate (HR) and respiration rate (RR). Of course, the processor module 1P can also calculate an estimated body temperature according to the body temperature sensing signal.

According to the present invention, the processor module 1P is further configured for generating a warning signal in case of there is existing said physical condition and/or at least one said physiological parameter exceeding a normal range, and then transmitting the warning signal to an electronic device 3 like the foregoing signal transceiver device through the wireless transmission interface 14W. Besides the signal transceiver device, the electronic device 3 can be a cloud server, a local server belong to a hospital, a postpartum center or an infant care center, and can also be a personal electronic device belong to the baby's parent, wherein the personal electronic device can be a tablet computer, a laptop computer, a desktop computer, an all-in-one computer, a smart phone, a smart watch, or a smart glasses.

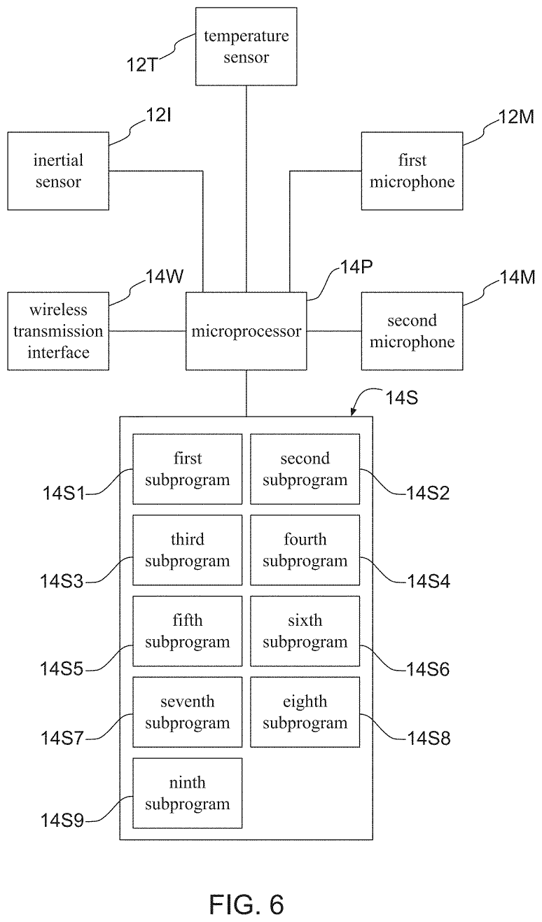

FIG. 6 shows a block diagram of the first microphone 12M, the temperature sensor 12T, the inertial sensor 12I, the second microphone 14M, the microprocessor 14P, the memory 14S, and the wireless transmission interface 14W they are shown in FIG. 5A and FIG. 5B. In one embodiment, the memory 14S stores an application program including instructions, such that in case the application program is executed, the microprocessor 14P is configured for controlling the first microphone 12M, the temperature sensor 12T, the inertial sensor 12I, the second microphone 14M, and the wireless transmission interface 14W, so as to achieve the measurement of physiological parameters (e.g., heart rate and respiratory rate) and the monitoring of the baby's physical conditions. As FIG. 6 shows, the application program consists of a plurality of subprograms, and the plurality of subprograms comprising: a first subprogram 14S1, a second subprogram 14S2, a third subprogram 14S3, a fourth subprogram 14S4, a fifth subprogram 14S5, a sixth 14S6, a seventh subprogram 14S7, an eighth subprogram 14S8, and a ninth subprogram 14S9. It is worth explaining that, not only does the memory 14S can be an embedded flash (eFlash) memory provided in the microprocessor 14P, but the memory 14S can also be a flash memory chip, a hard drive (HD), a solid state drive (SSD), or an USB flash drive that is coupled to the microprocessor 14P.

According to the present invention, the first subprogram 14S1 is compiled to be integrated in the application program by one type of programming language, and includes instructions for configuring the microprocessor 14P to control the temperature sensor 12T to measure a body temperature from the human body (e.g., a body 2 of a baby), thereby generating a body temperature sensing signal. Moreover, the second subprogram 14S2 is compiled to be integrated in the application program by one type of programming language, and includes instructions for configuring the microprocessor 14P to control the first microphone 12M and the second microphone 14M to collect a sound emitted from the human body, thereby generating a first sound signal and a second sound signal, respectively. In addition, the third subprogram 14S3 is compiled to be integrated in the application program by one type of programming language, and includes instructions for configuring the microprocessor 14P to control the inertial sensor 12I to monitor a movement and/or a vibration of the human body, thereby generating a body activity sensing signal. Furthermore, the fourth subprogram 14S4 is compiled to be integrated in the application program by one type of programming language, and includes instructions for configuring the microprocessor 14P to process the body temperature sensing signal, the first sound signal, the second sound signal, and/or the body activity sensing signal. As described in more detail below, the fifth subprogram 14S5 is compiled to be integrated in the application program by one type of programming language, and includes instructions for configuring the microprocessor 14P to apply a signal synchronizing process to the body temperature sensing signal, the first sound signal, the second sound signal, and the body activity sensing signal according to four timestamps that are respectively contained in the body temperature sensing signal, the first sound signal, the second sound signal, and the body activity sensing signal.

As FIG. 6 shows, the sixth 14S6 is compiled to be integrated in the application program by one type of programming language, and includes instructions for configuring the microprocessor 14P to judge whether the human body has at least one physical condition and then determine said physical condition. On the other hand, the seventh subprogram 14S7 is compiled to be integrated in the application program by one type of programming language, and includes instructions for configuring the microprocessor 14P to calculate an estimated body temperature according to the body temperature sensing signal, and to estimate at least one physiological parameter of the human body by processing the first sound signal, the second sound signal and the body activity sensing signal. In one practicable embodiment, the physiological parameter contains heart rate (HR) and/or respiration rate (RR). Moreover, the eighth subprogram 14S8 is compiled to be integrated in the application program by one type of programming language, and includes instructions for configuring the microprocessor 14P to judge whether there is a well contact between the first body 11 and the human body by analyzing the body temperature sensing signal, the body activity sensing signal, a first frequency band and a second frequency band of the first sound signal. According to the particular design of the present invention, before starting to monitor the physical conditions and/or measure the physiological parameters from the baby's body 2, the eighth subprogram 14S8 is executed by the microprocessor 14P, such that the microprocessor 14P is configured to judge whether there is a well contact between the first body 11 and the baby's body 2 or not. After the first body 11 is detected, by the sensor module 1S and the processor module 1P, to have already had a well contact with the baby's body 2, the microprocessor 14P immediately enables the real-time monitoring device 1 to start the measurement of physiological parameters (e.g., heart rate and respiratory rate) and the monitoring of the baby's physical conditions. By such design, the foregoing well-contact detecting function is not only helpful in making the real-time monitoring device 1 to achieve the measurement of physiological parameters and physical conditions of the baby with high accuracy, but also significantly save the power consumption of the real-time monitoring device 1. On the other hand, the ninth subprogram 14S9 is compiled to be integrated in the application program by one type of programming language, and includes instructions for configuring the microprocessor 14P to generate a warning signal in case of there is existing said physical condition and/or at least one said physiological parameter exceeding a normal range, and then to transmit a warning signal to the electronic device 3 through the wireless transmission interface 14W.

As described in more detail below, during the fact that the real-time monitoring device 1 works normally, the microprocessor 14P executes the first subprogram 14S1, such that the temperature sensor 12T is controlled to measure a body temperature from a human body (e.g., a body 2 of a baby), thereby generating a body temperature sensing signal. Simultaneously, the microprocessor 14P executes the second subprogram 14S2, such that the first microphone 12M and the second microphone 14M are controlled to collect a sound emitted from the baby's body 2, thereby generating a first sound signal and a second sound signal, respectively. Moreover, the microprocessor 14P also executes the third subprogram 14S3, such that the inertial sensor 12I is controlled to monitor the movement and/or a vibration of the baby's body 2, thereby generating a body activity sensing signal.

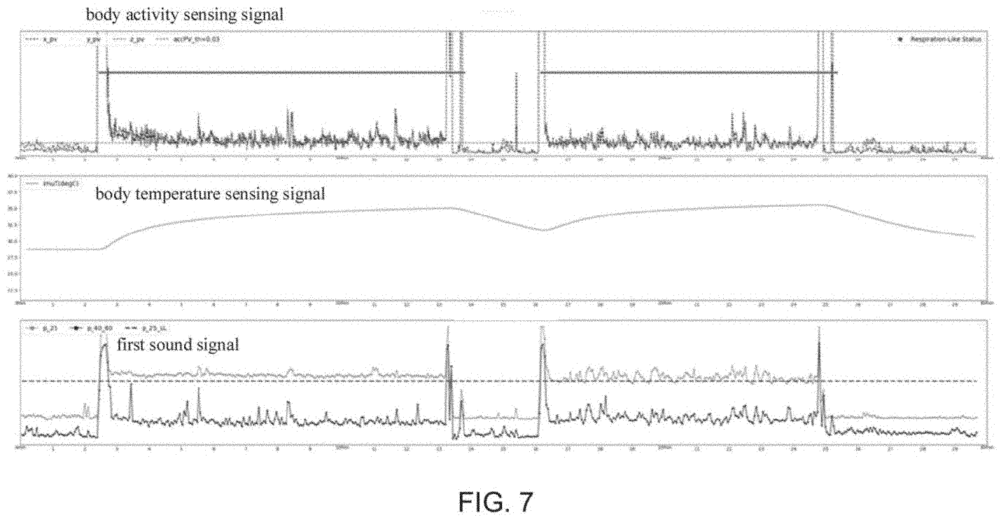

FIG. 7 shows a measured data graph of the body activity sensing signal, the body temperature sensing signal and the first sound signal. In case the eighth subprogram 14S8 is executed, the microprocessor 14P is configured to firstly determine whether the body activity sensing signal includes a signal segment for describing a breath variation of the baby. In other words, there is said signal segment in the body activity sensing signal means that the first body 11 of the sensor module 1S have already had a well contact with the baby's belly. On the other hand, it is also practicable to judge whether there is a well contact between the first body 11 and the baby's belly by analyzing the body temperature sensing signal. For example, in case the first body 11 is set to well contact the baby's belly by a body contacting surface, there is an obviously signal variation occurring in the body temperature sensing signal (as shown in FIG. 7). Of course, if there is a signal variation suddenly occurring in the body temperature sensing signal in case of the real-time monitoring device being operated, it means that the first body 11 is no longer having a well contact with the baby's belly. Particularly, it is also practicable to judge whether there is a well contact between the first body 11 and the baby's belly by analyzing the first sound signal. According to the measured data graph of FIG. 7, when the first body 11 is set to well contact the baby's belly, the magnitude of a first frequency band in the first sound signal shows an abruptly enhancement, wherein the first frequency band of the first sound signal includes the sound emitted from the baby's body 2 falls below the 25 Hz frequency band. Moreover, According to the measured data graph of FIG. 7, when the first body 11 is set to well contact the baby's belly, the magnitude of a second frequency band in the first sound signal also shows an abruptly enhancement, wherein the second frequency band of the first sound signal includes the sound emitted from the baby's body 2 falls between 40 Hz and 60 Hz.

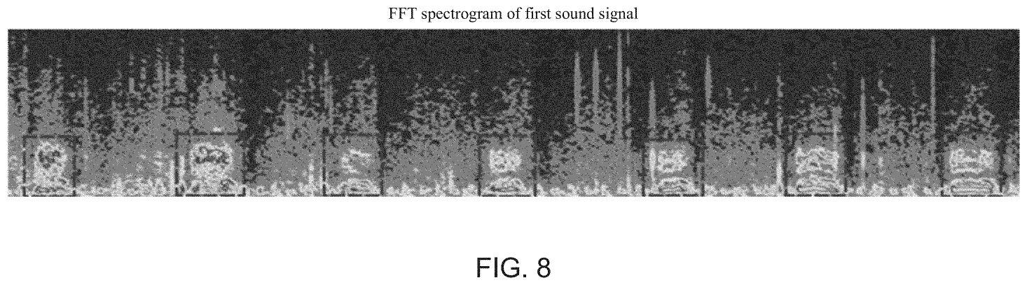

It needs to further explain that, the microprocessor 14P is provided with an analog-to-digital (A/D) convertor therein. After the microprocessor 14P receives the body activity sensing signal, the first sound signal and the second sound signal, the A/D convertor is enabled to directly digitize the first sound signal, digitize the second sound signal using a first sampling rate, and digitize the body activity sensing signal using a second sampling rate. In one embodiment, the first sampling rate is not greater than 4 KHz (i.e., ≤4 KHz), and the second sampling rate is not greater than 120 Hz (i.e., ≤120 Hz). After that, the microprocessor 14P executes the fourth subprogram 14S4, so as to process the first sound signal, the second sound signal, and/or the body activity sensing signal. For example, the microprocessor 14P apply a FFT (fast Fourier transform) process to the first sound signal and the second sound signal, thereby generating a first FFT spectrogram of the first sound signal and a second FFT spectrogram of the second sound signal. FIG. 8 illustrates a FFT spectrogram of the first sound signal containing airway obstruction feature. As FIG. 8 shows, after the first sound signal has received a FFT treatment, it is allowed to find out at least one signal segment containing airway obstruction feature(s) from the FFT spectrogram of the first sound signal.

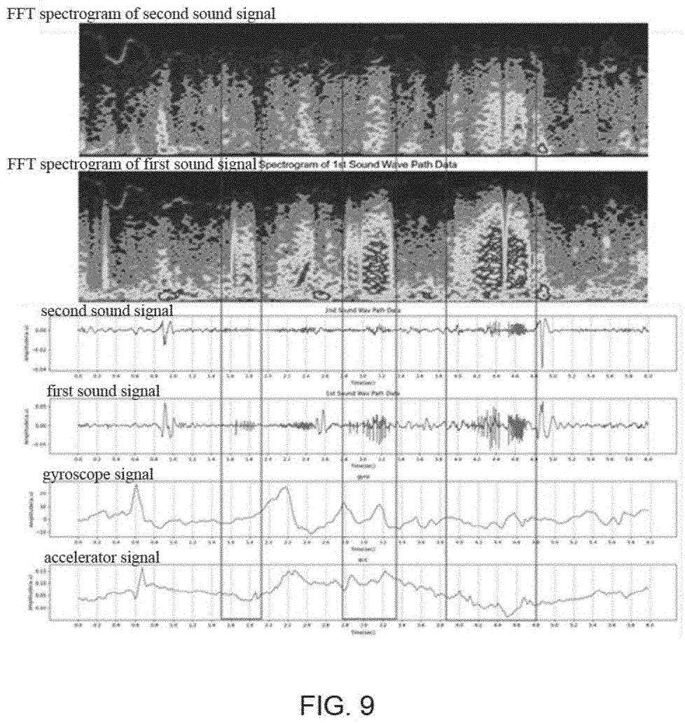

FIG. 9 illustrates a measured data graph of the first sound signal, the FFT spectrogram of the first sound signal, the second sound signal, the FFT spectrogram of the second sound signal, and body activity sensing signal. As FIG. 9 shows, by comparing the first sound signal with the second sound signal, it is able to know that a magnitude variation occurring in the first sound signal (i.e., the sound collected by the first microphone 12M from the baby's belly) is caused by environment noise, or is indeed a reflect of an inner sound of the baby's belly. Therefore, after said magnitude variation of the first sound signal is verified as a reflect of an inner sound of the baby's belly, the microprocessor 14P is subsequently configured to find out at least one signal segment containing abnormal bowel sound feature(s) from the first sound signal, the second sound signal and the body activity sensing signal (including gyroscope signal and accelerator signal). The segment containing abnormal bowel sound features are labeled by gray rectangular frame in FIG. 9.

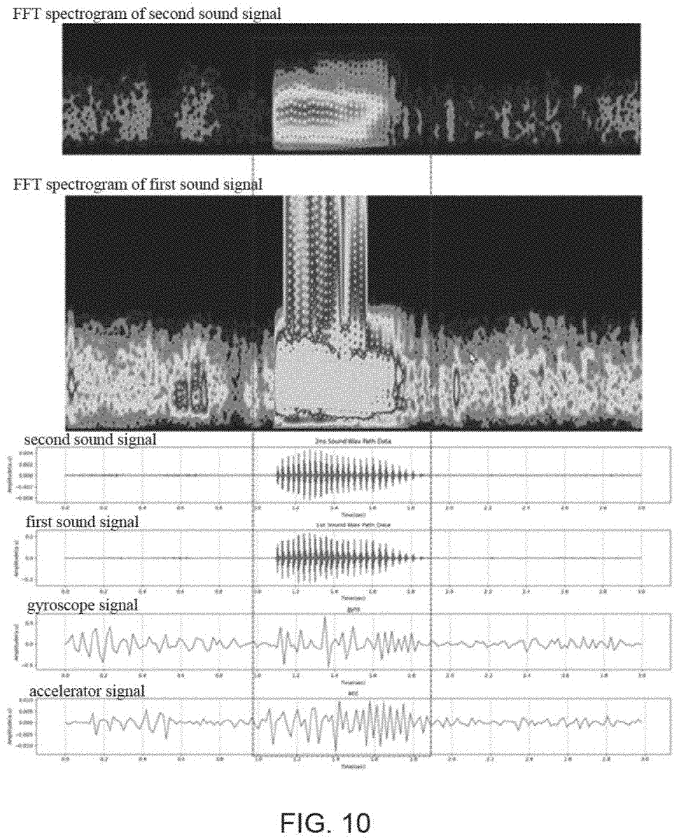

With reference to FIG. 10, there is a measured data graph of the first sound signal, the FFT spectrogram of the first sound signal, the second sound signal, the FFT spectrogram of the second sound signal, and body activity sensing signal provided. As FIG. 10 shows, by comparing the first sound signal with the second sound signal, it is able to know that a magnitude variation occurring in the first sound signal is caused by environment noise, or is indeed a reflect of an inner sound of the baby's belly. Subsequently, after said magnitude variation of the first sound signal is verified as a reflect of an inner sound of the baby's belly, the microprocessor 14P is configured to find out at least one signal segment containing excretion feature(s) from the first sound signal, the second sound signal and the body activity sensing signal (including gyroscope signal and accelerator signal). The segment containing excretion features are labeled by gray rectangular frame in FIG. 10.

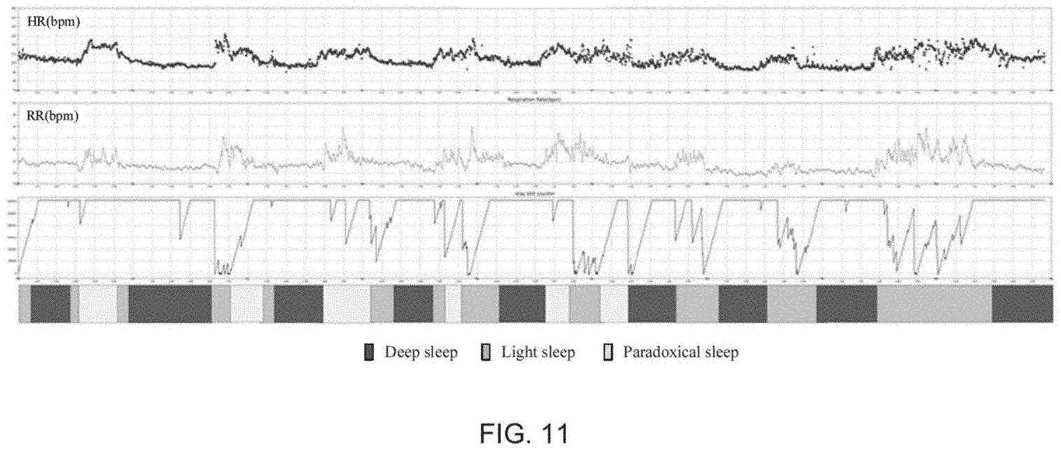

FIG. 11 illustrates a measured data graph of hearth rate signal and respiration rate signal. As FIG. 11 shows, after processing the body activity sensing signal, a hearth rate (HR) signal and respiration rate (RR) signal are therefore obtained. Next, it is able to find out at least one signal segment containing features of going into a deep sleep, going into a light sleep and going into a paradoxical sleep from the HR signal and the RR signal. Moreover, the microprocessor 14P can also estimates physiological parameters of the baby, including heart rate and respiration rate.

Therefore, through above descriptions, all embodiments and their constituting elements of the real-time monitoring device for human body according to the present invention have been introduced completely and clearly. Moreover, the above description is made on embodiments of the present invention. However, the embodiments are not intended to limit the scope of the present invention, and all equivalent implementations or alterations within the spirit of the present invention still fall within the scope of the present invention.

What is claimed is:

1. A real-time monitoring device for human body, comprising:
a sensor module, comprising a first body and a first circuit assembly disposed in the first body, wherein the first circuit assembly comprises a first microphone, a temperature sensor and an inertial sensor; and
a processor module, comprising a second body and a second circuit assembly disposed in the second body, wherein the second circuit assembly comprises a second microphone, a microprocessor, a memory, and a wireless transmission interface;
wherein the first body is allowed to be contacted a human body by a body contacting surface thereof, and the memory storing an application program including instructions, such that in case the application program is executed, the microprocessor being configured for:
controlling the temperature sensor to measure a body temperature from the human body, thereby generating a body temperature sensing signal;
controlling the first microphone to collect a sound emitted from the human body, thereby generating a first sound signal;
controlling the inertial sensor to monitor a movement and/or a vibration of the human body, thereby generating a body activity sensing signal;
controlling the second microphone to collect said sound emitted from the human body, thereby generating a second sound signal;
judging whether the human body has at least one physical condition by comparing the first sound signal with the second sound signal; and
analyzing the body temperature sensing signal, the first sound signal, the second sound signal, and the body activity sensing signal, so as to determine said physical condition includes at least one selected from a group consisting of excretion, abnormal heart rate (HR), abnormal respiration rate (RR), emission of abnormal bowel sounds, airway obstruction, going into a deep sleep, going into a light sleep, and going into a paradoxical sleep.

2. The real-time monitoring device for human body of claim 1, wherein the application program consists of a plurality of subprograms, and the plurality of subprograms comprising:
a first subprogram, being compiled to be integrated in the application program by one type of programming language, and including instructions for configuring the microprocessor to control the temperature sensor to measure the body temperature from the human body;
a second subprogram, being compiled to be integrated in the application program by one type of programming language, and including instructions for configuring the microprocessor to control the first microphone and the second microphone to collect the sound emitted from the human body;
a third subprogram, being compiled to be integrated in the application program by one type of programming language, and including instructions for configuring the microprocessor to control the inertial sensor to monitor the movement and/or the vibration of the human body;
a fourth subprogram, being compiled to be integrated in the application program by one type of programming language, and including instructions for configuring the microprocessor to process the body temperature sensing signal, the first sound signal, the second sound signal, and/or the body activity sensing signal;
a fifth subprogram, being compiled to be integrated in the application program by one type of programming language, and including instructions for configuring the microprocessor to apply a signal synchronizing process to the body temperature sensing signal, the first sound signal, the second sound signal, and the body activity sensing signal according to four timestamps that are respectively contained in the body temperature sensing signal, the first sound signal, the second sound signal, and the body activity sensing signal; and
a sixth, being compiled to be integrated in the application program by one type of programming language, and including instructions for configuring the microprocessor to judge whether the human body has at least one physical condition and then determine said physical condition.

3. The real-time monitoring device for human body of claim 2, wherein the plurality of subprograms further comprises:
a seventh subprogram, being compiled to be integrated in the application program by one type of programming language, and including instructions for configuring the microprocessor to calculate an estimated body temperature according to the body temperature sensing signal, and to estimate at least one physiological parameter of the human body by processing the first sound signal, the second sound signal and the body activity sensing signal; wherein the physiological parameter is selected from a group consisting of heart rate (HR) and respiration rate (RR).

4. The real-time monitoring device for human body of claim 3, wherein the plurality of subprograms further comprises:
an eighth subprogram, being compiled to be integrated in the application program by one type of programming language, and including instructions for configuring the microprocessor to judge whether there is a well contact between the first body and the human body by analyzing the body temperature sensing signal, the body activity sensing signal, a first frequency band and a second frequency band of the first sound signal.

5. The real-time monitoring device for human body of claim 4, wherein the plurality of subprograms further comprises:
a ninth subprogram, being compiled to be integrated in the application program by one type of programming language, and including instructions for configuring the microprocessor to generate a warning signal in case of there is existing said physical condition and/or at least one said physiological parameter exceeding a normal range, and then to transmit the warning signal to an electronic device through the wireless transmission interface.

6. The real-time monitoring device for human body of claim 5, wherein the electronic device is selected from a group consisting of signal transceiver device, tablet computer, cloud server, laptop computer, desktop computer, all-in-one computer, smart phone, smart watch, and smart glasses.

7. The real-time monitoring device for human body of claim 5, wherein the memory is selected from a group consisting of embedded flash (eFlash) memory, flash memory chip, hard drive (HD), solid state drive (SSD), and USB flash drive.

8. The real-time monitoring device for human body of claim 5, wherein the microprocessor is provided with an analog-to-digital (A/D) convertor therein, and the A/D convertor directly digitizing the first sound signal, digitizing the second sound signal using a first sampling rate, and digitizing the body activity sensing signal using a second sampling rate.

9. The real-time monitoring device for human body of claim 8, wherein the first sampling rate is not greater than 4 KHz, and the second sampling rate being not greater than 120 Hz.

10. The real-time monitoring device for human body of claim 1, wherein the first body has a first accommodation space for receiving the first circuit assembly therein, and a first cover being connected to a first opening of the first accommodation space so as to shield the first circuit assembly.

11. The real-time monitoring device for human body of claim 10, wherein an aperture being formed on a bottom of the first accommodation space, such that the first microphone is exposed out of the first body via the aperture.

12. The real-time monitoring device for human body of claim 11, wherein a circular recess is formed on the body contacting surface of the first body, and the circular recess having a depth and a diameter in a range between 4.5 mm and 20 mm, such that a ratio of the diameter to the depth being not greater than 6.

13. The real-time monitoring device for human body of claim 12, wherein a minimum value of the depth is 1.5 mm.

14. The real-time monitoring device for human body of claim 12, wherein the second body has a second accommodation space for receiving the second circuit assembly therein, and a second cover being connected to a second opening of the second accommodation space so as to shield the second circuit assembly.

15. The real-time monitoring device for human body of claim 12, wherein a body connecting member is connected between the first body and the second body, and the body connecting member being provided with an electrical connecting component therein, such that the first circuit assembly is coupled to the second circuit assembly through the electrical connecting component.

16. The real-time monitoring device for human body of claim 15, further comprising:
an article supporting unit, being disposed in the second accommodation space, and consisting of a platform and a plurality of supporting rods; wherein the platform is faced to a bottom of the second accommodation space, and the second circuit assembly being positioned in a space formed by the plurality of supporting rods and a bottom surface of the platform.

17. The real-time monitoring device for human body of claim 16, wherein the processor module further comprises:
a wireless charging module, being disposed on a top surface of the platform, and being coupled to the second circuit assembly; and
a battery, being coupled to the second circuit assembly.

18. The real-time monitoring device for human body of claim 15, wherein the second body, the body connecting member 1B and the first body are allowed to be fixed on a mounting kit, such that after disposing the mounting kit on an article that is worn on the human body, the first body being set to contact the human body by the body contacting surface thereof.

19. The real-time monitoring device for human body of claim 18, wherein in case of the first body being set to contact the human body, a device fixing member is allowed to be used in further fixing the second body on the article.

20. The real-time monitoring device for human body of claim 15, wherein the second body and the first body are allowed to be connected with a device fixing member, such that the second body and the first body are allowed to be attached onto the human body through the device fixing member, thereby making the first body contact the human body by the body contacting surface thereof.
